(19) United States
(12) Patent Application Publication
(10) Pub. No.: US 2012/0089219 A1
(43) Pub. Date: Apr. 12, 2012

(54) STENT HAVING INCREASED VISIBILITY IN THE X-RAY IMAGE

(75) Inventors: Horst Fircho, Koesterbeck (DE); Frank Bakczewitz, Rostock (DE); Markus Wolfer, Kleinandelfingen (CH)

(73) Assignee: BIOTRONIK AG, Buelach (CH)

(21) Appl. No.: 13/248,667

(22) Filed: Sep. 29, 2011

Related U.S. Application Data
(60) Provisional application No. 61/391,087, filed on Oct. 8, 2010.

(51) Int. Cl. A61F 2/82

(52) U.S. Cl. 623/1.16; 623/1.34

(57) ABSTRACT

A stent comprising a tubular base body having a lumen along a longitudinal axis. The base body has a plurality of circumferential support structures and one or more connectors. Two successive circumferential support structures are connected to one another via at least one connector. The stent is characterized in that one, multiple, or all support structures and/or connectors have a slotted passage, and the slotted passage is filled with a radiopaque material and has a curvature which increases at least toward one end of the slotted passage.

### CROSS REFERENCE TO RELATED APPLICATION

This patent application claims the benefit of U.S. Provisional Patent Application No. 61/391,087, filed on Oct. 8, 2010, which is hereby incorporated by reference in its entirety.

### TECHNICAL FIELD

The invention relates to a medical implant, in particular a stent.

### BACKGROUND

The implantation of stents has become established as one of the most effective therapeutic measures in the treatment of vascular diseases. Stents perform a support function in hollow organs of a patient. For this purpose, stents of conventional design have a base body which has numerous circumferential support structures made of metallic braces, for example, which for insertion into the body are initially in a compressed form, and are then expanded at the site of application. One of the main fields of application of such stents is to permanently or temporarily widen and keep open vascular constrictions, in particular constrictions (stenoses) of the coronary vessels. In addition, aneurysm stents, for example, are also known which are used for supporting damaged vascular walls.

Stents have a tubular base body of sufficient load capacity to keep the constricted vessel open to the desired extent, through which blood flows through unhindered. The circumferential wall of the base body is generally formed by a lattice-like support structure which allows the stent to be inserted in a compressed state, with a small outer diameter, up to the constriction to be treated in the particular vessel, and at that location, for example by use of a balloon catheter, to be expanded until the vessel has the desired enlarged inner diameter. The process of positioning and expanding the stent during the procedure and the subsequent location of the stent in the tissue after the procedure is completed must be monitored by the cardiologist. This may be achieved using imaging methods such as X-ray analysis, for example.

The stent itself is usually made of materials which are not sufficiently radiopaque to be representable in the desired quality using imaging X-ray analysis. That is, the stent is usually provided with a radiopaque material, a suitable X-ray marker, in order to be representable in the X-ray image.

At the present time, such X-ray markers are usually either applied to the stent as end-position, planar coatings, or fastened to the stent at specific points in a microriveting process, for example. Radiopaque materials are used which contain or are composed of Au, Pt, and/or Ta, for example. This type of marking of stents with radiopaque markers has the disadvantage that the radiopacity is relatively low, and is usually highly influenced by the beam path, and therefore is dependent on position.

The radiopacity is particularly relatively low for end-position, planarly coated stents due to the small layer thickness. In addition, so-called “dog boning” effects occur in angiography; i.e., the ends illuminate the image so as to give the impression that the ends of the stent are open farther than the middle stent region. As a result, in the regions of the stent which are subjected to deformation there is a risk that the coating with the X-ray will come off and be lost.

On the other hand, the microriveting process requires complex equipment, is costly, and may result in a reject rate which is not negligible. In addition, the stent architecture and the crimp design must be selected in such a way that the ends of the stent provide sufficient space for accommodating the cylindrical rivets.

The object of the present invention is to reduce or avoid one or more of the disadvantages of the prior art.

The object is achieved by providing a stent comprising a tubular base body having a lumen along a longitudinal axis, the base body having a plurality of circumferential support structures and one or more connectors, and two successive circumferential support structures being connected to one another via at least one connector, characterized in that one, multiple, or all support structures and/or connectors have a slotted passage, the slotted passage being filled with a radiopaque material and having a curvature which increases at least toward one end of the slotted passage.

Slotted passages are introduced into the components of the base structure of the stent according to the invention, in particular into the support structures and/or connectors, and penetrate the web of the support structures and/or connectors from the outer surface to the inner surface. These slotted passages are filled with a radiopaque material. Similarly as for the use of microrivets, a very high radiopacity is achieved by applying greater quantities of radiopaque material compared to the end-position coating. In contrast to the use of planar coatings, the contact area between the radiopaque material and the vessel wall or the bloodstream is very small. The slotted passage has a curvature which is not the same over the entire length of the slot, but which instead increases at least toward one end of the slotted passage. Thus, not only is the radiopaque material in contact with the material of the base body of the stent over a comparatively greater surface area, but the radiopaque material is also better mechanically fixed due to the various curvatures of the slotted passage.

As the result of the radiopaque material being fixed in the slotted passage via simultaneous frictional and form fit, particularly stable integration of the radiopaque material into the stent is ensured, also in comparison to the use of microrivets, thus greatly reducing the likelihood of loss of X-ray marker material during use of the stent. In addition to the mechanical stability, the stent according to the invention is characterized in that it may be manufactured using less complex methods such as laser cutting and deposition of radiopaque material, so that the high level of complexity and reject rate of the microriveting process may be avoided.

The arc-shaped curved geometry of the markers has the additional advantage that the dependency on position of other marker geometries (rivets, for example) is reduced due to the presence of a great marker thickness and therefore absorption of the X-rays in all positions, thus improving the visibility.

In principle, the slotted passages may be provided at any location on the base body of the stent. Compared to end-position markers (typically for rivets and surface area coatings), this has the advantage that the stent may be positioned very precisely when used in the region of bifurcations, since the stent position with respect to the branch in the angiography may be accurately determined by suitable placement of the slotted markers in the middle longitudinal region of the stent.

The stent according to the invention has a tubular base body which encloses a lumen along a longitudinal axis of the stent. Blood is able to flow through this lumen after installation of the stent in a blood vessel.

The base body of the stent according to the invention includes a plurality of circumferential support structures which are successively situated along the longitudinal axis of the stent and enclose the lumen. The support structures are each made up of a consecutive series of diagonal elements and arched elements (also referred to as crowns), each of which may be formed from braces made of an implant material. The diagonal elements have an elongated shape having two ends, and connect two arched elements having opposite curvatures. The diagonal elements are essentially responsible for extending a support structure in the direction of the longitudinal axis. The arched elements are curved, and connect two successive diagonal elements of a support structure to one another in such a way that the latter come to rest one on top of the other along an axis extending vertically with respect to the longitudinal axis, resulting in an annular circumferential structure which encloses a lumen.

The base body of the stent according to the invention includes one or more connectors in addition to a plurality of support structures, whereby two successive circumferential support structures are connected to one another via at least one connector. On the one hand, these connectors must be situated in such a way that they ensure sufficient bending flexibility of the stent, and on the other hand they must not hinder a crimping and/or dilation process. The connectors of the stent according to the invention are designed in such a way that a plurality of support structures may be connected to a base body which is suitable for use in an expandable stent. For this purpose, in each case a connector is connected at a first end to a diagonal element of a first support structure, and at a second end is connected to a diagonal element of a second support structure. Two successive support structures may also be connected to one another via more than one connector. One, multiple, or all connectors of the stent according to the invention may have an elongated shape with two opposite ends. The connectors may be formed from braces made of an implant material. The connectors are preferably only long enough to ensure sufficient flexibility of the two adjacent support structures, but not so long that the stent according to the invention becomes torsionally flexible. One, multiple, or all connectors of a stent according to the invention may have a curved shape. One, multiple, or all connectors of a stent according to the invention may each branch off from the diagonal element at an acute angle. The connectors are essentially oriented in the direction of the longitudinal axis, between the two circumferential support structures to be connected, whereby the connectors are not necessarily aligned in parallel with respect to the longitudinal axis.

The stent according to the invention has one or more slotted passages which are filled with a radiopaque material. In principle, these slotted passages may be provided at any location on the base body of the stent.

In particular, one, multiple, or all support structures and/or connectors of the base body of the stent have one or more slotted passages. The slotted passages extend from the outer surface of the support structure and/or of the connector facing the lumen of the base body and through the entire brace, up to and including the outer surface of the support structure and/or of the connector facing away from the lumen of the stent. Thus, the slotted passage completely penetrates the brace of the support structure or of the connector. The passage extends in a slotted shape, parallel to the outer surface of the support structure or of the connector, and is present in this plane as an oblong opening having two opposite ends. The distance between the two opposite ends specifies the maximum length/of the opening. The distance between the two walls connecting the opposite ends at the widest location of the opening specifies the maximum width b. The oblong opening in the slotted passage preferably has a ratio of the maximum length 1 to the maximum width b of the opening of ≧2, particularly preferably ≧5, very particularly preferably ≧10.

The slotted passage has a curvature over the maximum length/of the oblong opening which increases at least toward one end of the slotted passage. This ensures that the curvature is not uniform over the entire oblong opening in the slotted passage, but instead, that the slotted passage has regions of different curvatures. A tension is thus produced which results in improved mechanical fixing of the radiopaque material in the slotted passage.

The mechanical fixing of the radiopaque material in the slotted passage may be further improved as the result of the slotted passage having a curvature which increases toward both opposite ends of the oblong opening in the slotted passage. In the present case, an “increase” is understood to mean any deviation toward a larger numerical value, independent of the algebraic sign (and thus the direction) of the particular curvature. The direction of the particular curvature at the two opposite ends may be the same or different. In particular, the direction of the curvature over the progression of the maximum length/of the oblong opening in the slotted passage may alternate multiple times. Thus, the curvature may have a meandering shape, for example.

In one preferred embodiment, the shape of the curvature in the region of the two opposite ends of the oblong opening in the slotted passage is essentially uniform or oppositely directed. In particular, the curvature may be such that the shape of the oblong opening, and preferably the design of the opposite ends, has an axis of symmetry or a point of symmetry.

The shape of the curvature in particular may be selected in such a way that the oblong opening in the slotted passage in the region of the opposite ends has a curved, hooked, or spiral design. Preferred embodiments are schematically illustrated by way of example in FIGS. 1 through 7. In FIG. 1 the two opposite ends are designed as uniform hooks. In this case the oblong opening is axially symmetrical with respect to sectional plane A-A. In FIG. 2 the two opposite ends are designed as oppositely directed hooks. As shown in FIG. 3, a slotted passage may have a corresponding curvature only at one end. A component of the base body of the stent may have more than one slotted passage, whereby the slotted passages may have a design that is uniform (FIG. 3) or oppositely directed (FIG. 4). The shape of the components of the base body may be specifically adapted and/or designed for accommodating a slotted passage. Thus, the component of the base framework of the stent in FIG. 5 has a projection which is used to accommodate a slotted passage in the form of a spiral. FIGS. 6 and 7 show alternative embodiments of the base framework geometry which are specifically adapted for accommodating slotted passages.

The slotted passage of the stent according to the invention is filled with a radiopaque material. The radiopaque material is characterized in that it may be represented in an imaging X-ray analysis. In this regard, radiopaque materials which are used in known X-ray markers may be employed. In particular, the radiopaque material contains or is composed of Au, Pt, and/or Ta, or an alloy or mixture thereof. Suitable radiopaque materials are known to those skilled in the art.

Since in the stent according to the invention the radiopaque material is in contact with the base body of the stent over a relatively large surface of the slotted passage, and in addition the selection of the curvature assists in mechanical fixing of the radiopaque material in the passage, the filling together with the radiopaque material may form a flat closure with the outer surface of the particular component of the base body of the stent. This has the advantage that protrusions of radiopaque material are not able to project into the lumen of the stent or the surface of the base body of the stent to be contacted by the vessel wall which may result in turbulence, injuries, or other disturbances at those locations. The useful surfaces of the base body remain flat while ensuring sufficient fixing of the radiopaque material in the slotted passage. FIG. 8 schematically shows a top view of the cut surface of the sectional plane along cutting guide A-A from FIG. 1. It is apparent that the filling with the radiopaque material on both sides of the slotted passage ends at the outer surface of the component of the base framework in such a way that a flat outer surface results. The filling with radiopaque material is fixed in a form-fit manner in the slotted passage on account of the surface roughness of the inner walls of the slotted passage.

Alternatively, the filling with a radiopaque material may form a convex closure with the outer surface of the support structure and/or of the connector which surrounds the slotted passage. The mechanical fixing of the radiopaque material in the base body of the stent may be further increased in this way. The convex closure may preferably have a design which projects beyond the edge of the outer surface. The edges of the outer surface of the support structure and/or of the connector particularly preferably have a rounded design, so that the protrusion of the filling beyond the face of the rounded edges comes into contact with the outer surface. This has the advantage that breakage of the protrusion at the edge of the outer surface is less likely. A corresponding design is illustrated by way of example in FIG. 9. The form-fit fixing results not only from the roughness of the inner surfaces of the slotted passage, but also from the protrusions beyond the rounded edges of the outer surfaces of the support structure and/or of the connector provided on both sides.

The base body of the stent according to the invention may be formed from an implant material. An implant material is a nonliving material which is used for medical applications and interacts with biological systems. The basic requirement for use of a material as an implant material, which when properly used is in contact with the bodily surroundings, is compatibility with the body (biocompatibility). Biocompatibility is understood to mean the ability of a material to induce an appropriate tissue reaction in a specific application. This includes adaptation of the chemical, physical, biological, and morphological surface characteristics of an implant to the recipient tissue, with the objective of a clinically sought interaction. The biocompatibility of the implant material is also dependent on the time sequence of the reaction of the biosystem which has received the implant. Relatively short-term irritation and inflammation occur which may result in changes in the tissue. Accordingly, biological systems react in various ways, depending on the characteristics of the implant material. The implant materials may be divided into bioactive, bioinert, and degradable/absorbable materials, depending on the reaction of the biosystem.

The base body of the stent according to the invention may be composed of any implant material that is suitable for the manufacture of implants, in particular stents. Implant materials for stents include polymers, metallic materials, and ceramic materials. Biocompatible metals and metal alloys for permanent implants contain, for example, stainless steel (316L, for example), cobalt-based alloys (CoCrMo cast alloys, CoCrMo forged alloys, CoCrWNi forged alloys, and CoCrNiMo forged alloys, for example), pure titanium and titanium alloys (CP titanium, TiAl6V4, or TiAl6Nb7, for example), and gold alloys.

The base body preferably contains a metallic implant material or is composed of same.

The stent according to the invention particularly preferably has a base body which contains a biodegradable implant material or is composed of same. For biocorrodible stents the use of magnesium or pure iron, or biocorrodible base alloys of the elements magnesium, iron, zinc, molybdenum, and tungsten, is recommended. In particular, the base body of a stent according to the invention may contain a biocorrodible magnesium alloy or be composed of same.

“Alloy” is understood herein to mean a metallic structure having magnesium, iron, zinc, or tungsten as its main component. The main component is the alloy component having the highest proportion by weight in the alloy. A proportion of the main component is preferably greater than 50% by weight, in particular greater than 70% by weight.

The composition of alloys of the elements magnesium, iron, zinc, or tungsten may be selected so as to be biocorrodible. Within the meaning of the invention, “biocorrodible” refers to alloys for which, in a physiological environment, degradation occurs which ultimately results in the entire implant or the part of the implant formed from the material losing its mechanical integrity. Synthetic plasma as specified according to EN ISO 10993-15:2000 for biocorrosion testing (composition: NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l) is a suitable test medium for testing the corrosion behavior of a given alloy. A sample of the alloy to be tested is accordingly stored together with a defined quantity of the test medium in a sealed sample container at 37° C. At time intervals of a few hours to several months, depending on the expected corrosion behavior, the samples are removed and investigated in a known manner for signs of corrosion. The synthetic plasma according to EN ISO 10993-15:2000 corresponds to a medium similar to blood, and therefore provides the possibility for reproducibly representing a physiological environment within the meaning of the invention.

The term “corrosion” herein refers to the reaction of a metallic material with its environment whereby, when the material is used in a component, a measurable change in the material causes impairment of the function of the component. In the present context a corrosion system is composed of the corroding metallic material and a liquid corrosion medium whose composition reproduces the conditions in the physiological environment, or which is a physiological medium, in particular blood. Material factors which influence the corrosion include the composition and pretreatment of the alloy, microscopic and submicroscopic inhomogeneities, boundary zone characteristics, temperature and stress state, and in particular the composition of a layer covering the surface. With regard to the medium, the corrosion process is influenced by conductivity, temperature, temperature gradients, acidity, volume-surface ratio, concentration difference, and flow velocity.

DE 197 31 021 A1 discloses suitable biocorrodible metallic implant materials having an element from the group of alkali metals, alkaline earth metals, iron, zinc, and aluminum as their main component. Alloys based on magnesium, iron, and zinc are described as being particularly suitable. Secondary components of the alloys may include manganese, cobalt, nickel, chromium, copper, cadmium, lead, tin, thorium, zirconium, silver, gold, palladium, platinum, silicon, calcium, lithium, aluminum, zinc, and iron. Furthermore, from DE 102 53 634 A1 the use of a biocorrodible magnesium alloy is known, having a proportion of magnesium >90%, yttrium 3.7-5.5%, rare earth metals 1.5-4.4%, and the remainder <1%, which is particularly suited for manufacturing a stent, for example in the form of a self-expanding or balloon-expandable stent.

The stent according to the invention may be manufactured, for example, by incorporating the slotted passages into the base body in the desired curvature and shape, for example using laser cutting or laser milling processes, after the stent base body is produced. The radiopaque material may then be applied. This may be carried out using deposition processes, for example, wherein radiopaque material is deposited until the slotted passages are completely filled with radiopaque material. Excess radiopaque material may then be removed from the base body of the stent.

### DESCRIPTION OF THE DRAWINGS

The invention is explained in greater detail below with reference to exemplary embodiments.

FIG. 1 schematically shows a detail of the base framework of a stent according to the invention, the illustrated component of the base framework having a slotted passage in the region of the arched element which is filled with radiopaque material, wherein the two opposite ends of the slotted passage are designed as hooks having uniform shapes of curvature. In the section along sectional plane A-A it can be seen that the slotted passage completely penetrates the brace of the arched element.

FIG. 2 schematically shows a detail of the base framework of a stent according to the invention, the illustrated component of the base framework having a slotted passage in the region of the arched element which is filled with radiopaque material, wherein the two opposite ends of the slotted passage are designed as hooks having oppositely directed shapes of curvature.

FIG. 3 shows another embodiment of the stent according to the invention, the illustrated component of the base framework having two slotted passages. Each of the two slotted passages has a corresponding hook-shaped curvature only in the region of one of the two ends, wherein the shapes of the curvatures of the two slotted passages are uniform.

FIG. 4 shows another embodiment of the stent according to the invention, the illustrated component of the base framework having two slotted passages. Each of the two slotted passages has a corresponding hook-shaped curvature only in the region of one of the two ends, wherein the shapes of the curvatures of the two slotted passages are oppositely directed.

FIG. 5 schematically shows a detail of the base framework of a stent according to the invention, and in the region of the arched element the illustrated component of the base framework has a projection having a slotted passage which is filled with radiopaque material, wherein the slotted passage has a spiral design.

FIG. 6 schematically shows a detail of the base framework of a stent according to the invention, the illustrated component of the base framework having a thickened region having a slotted passage which is filled with radiopaque material, wherein the two opposite ends of the slotted passage are designed as oppositely directed spirals.

FIG. 7 shows another embodiment of the stent according to the invention, the illustrated component of the base framework having two thickened regions, each having a slotted passage. Each of the two slotted passages has a corresponding hook-shaped curvature only in the region of one of the two ends, wherein the shapes of the curvatures of the two slotted passages are uniform.

FIG. 8 shows a schematic top view of the cut surface of the sectional plane along cutting guide A-A from FIG. 1. It is apparent that the filling with the radiopaque material on both sides of the slotted passage ends at the outer surface of the component of the base framework in such a way that a flat outer surface results. The filling with radiopaque material is fixed in a form-fit manner in the slotted passage on account of the surface roughness of the inner walls of the slotted passage.

FIG. 9 shows a schematic top view of the cut surface of the sectional plane along cutting guide A-A from FIG. 1. The form-fit fixing of the filling with the radiopaque material results not only from the roughness of the inner surfaces of the slotted passage, but also from the protrusions of the filling beyond the rounded edges of the outer surfaces of the support structure and/or of the connector provided on both sides.

### DETAILED DESCRIPTION

### Exemplary Embodiments

### Exemplary Embodiment 1

In a commercially available stent, slotted passages are incorporated into the braces of the base body using laser cutting processes. The stent having the slotted passages is then electropolished and ultrasonically cleaned in preparation for the electroplating deposition of, for example, Pt and Au, and optionally Ta by use of ionic liquid. Using an electroplating process sequence (gold bonding process, gold paste coating), the slotted passages and the planar enclosure of the braces are coated with the marker material, for example Au, Pt, Ta, using customized process parameters, as the result of which the slotted passage is completely filled with the marker material in a bonding and form-fit manner.

The filled slotted passages may then be lithographically covered and/or protected for a subsequent “stripping” process step in which the radiopaque material goes into solution outside the slotted region and is thus removed.

The end result is a stent having a radiopaque marker which may be subjected to high mechanical stresses in a bonding and form-fit manner without microcracks or peeling occurring.

### Exemplary Embodiment 2

In a commercially available stent, slotted passages are incorporated into the braces of the base body using laser cutting processes. The stent having the slotted passages is then electropolished and ultrasonically cleaned in preparation for the electroplating deposition of, for example, Pt and Au, and optionally Ta by use of ionic liquid. Using an electroplating process sequence (gold bonding process, gold paste coating), the slotted passages and the planar enclosure of the braces are coated with the marker material, for example Au, Pt, Ta, using customized process parameters, as the result of which the slotted passage is completely filled with the marker material in a bonding and form-fit manner. The “stripping” process step is omitted.

The end result is a stent having a radiopaque marker which may be subjected to high mechanical stresses in a bonding and form-fit manner without microcracks or peeling occurring.

### Exemplary Embodiment 3

The segments of both ends of the stent are lithographically covered using a UV/laser reactive protective lacquer. The areas of the stent ends at which slotted passages are to be incorporated are exposed and cleaned in a subsequent “developing” process step, using UV or laser exposure. The exposed and cleaned slotted passages may then be completely filled with the radiopaque material in a coating process using plating technology. Depending on the process parameters, the end-face surface of the filling in the slotted passages may be provided with a design which is convex, concave, or in flush alignment with respect to the outer surface of the surrounding braces.

The end result is a stent having radiopaque markers, having adapted topological transitions and without sharp edges with respect to the surroundings at the same level, which may be subjected to high mechanical stresses in a bonding and form-fit manner without microcracks or peeling occurring.

### Exemplary Embodiment 4

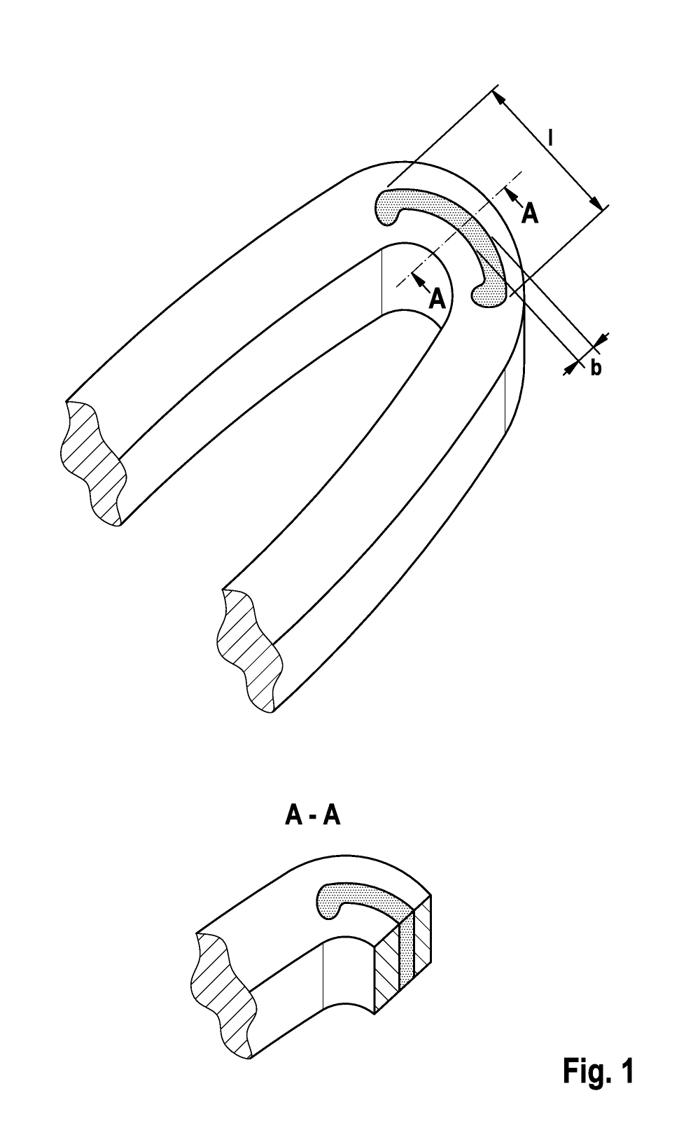

The slotted passage may be implemented in many different forms and variants. Thus, as shown in FIG. 1, the slotted passage may be designed, for example, in such a way that the two opposite ends are provided as hooks, each having a uniform shape of curvature. In the illustration of the section along cutting guide A-A it is obvious that the slotted passage extends through the entire brace and completely penetrates same. In the top view the slotted passage has an oblong opening. The oblong opening in the slotted passage has a ratio of the maximum length l to the maximum width b of ≧2.

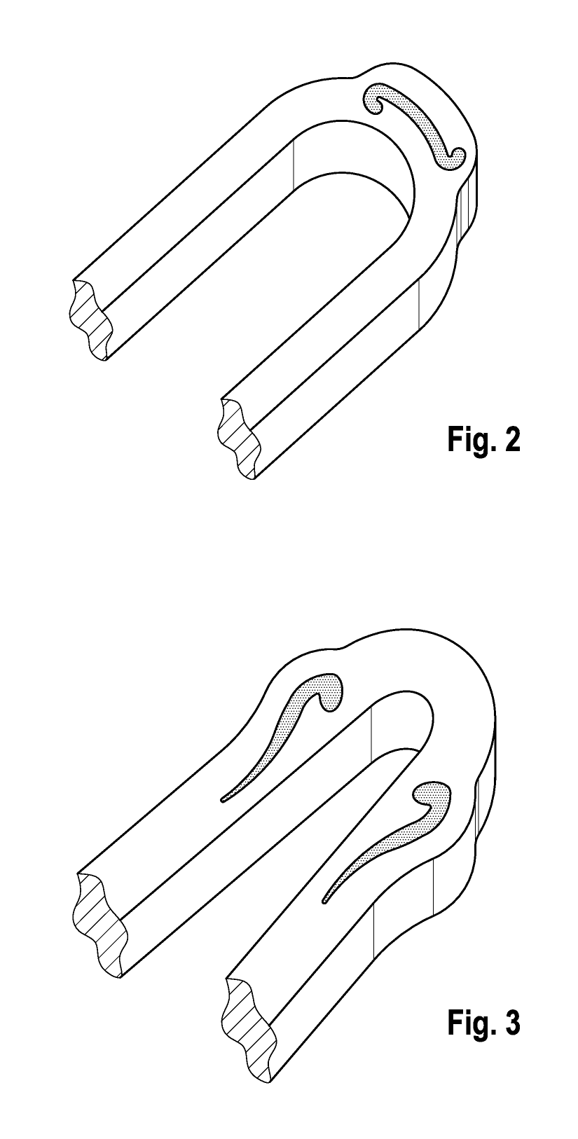

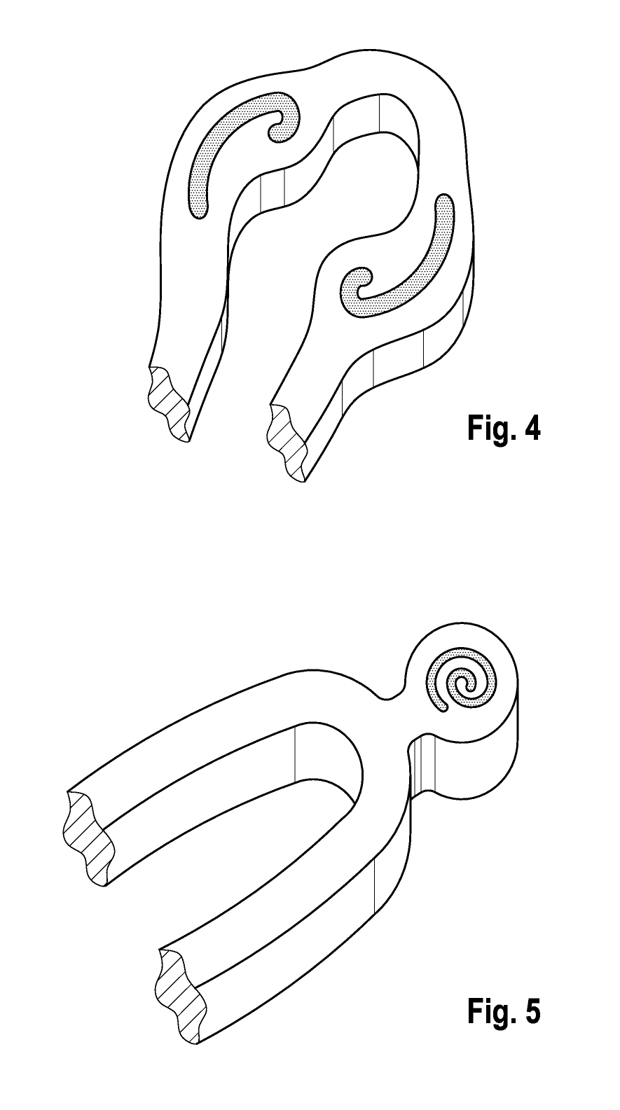

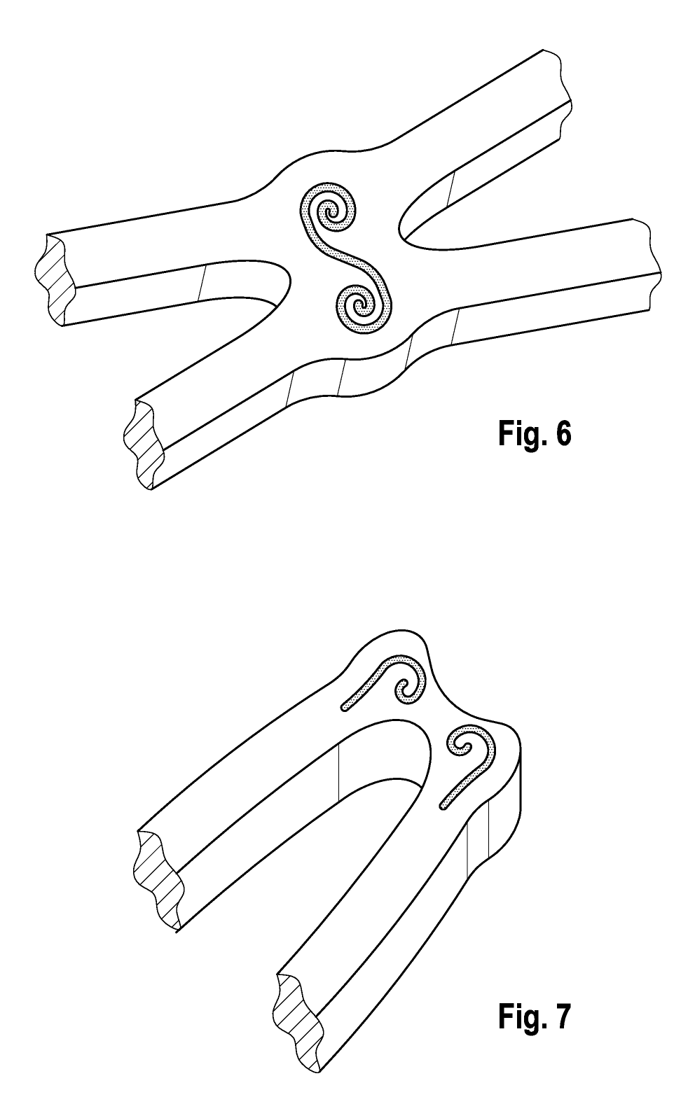

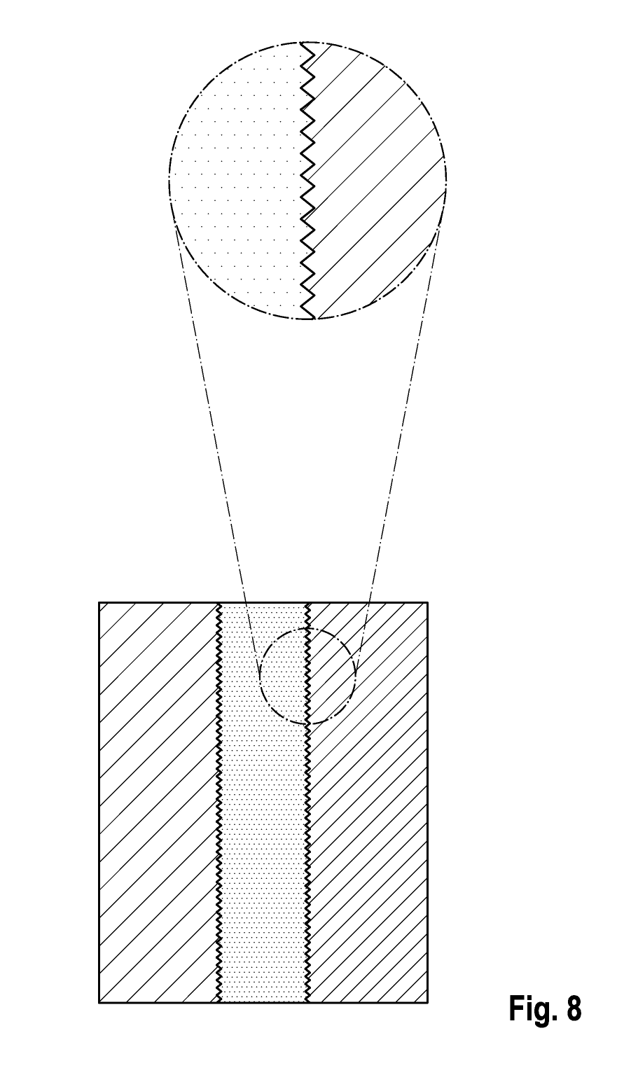

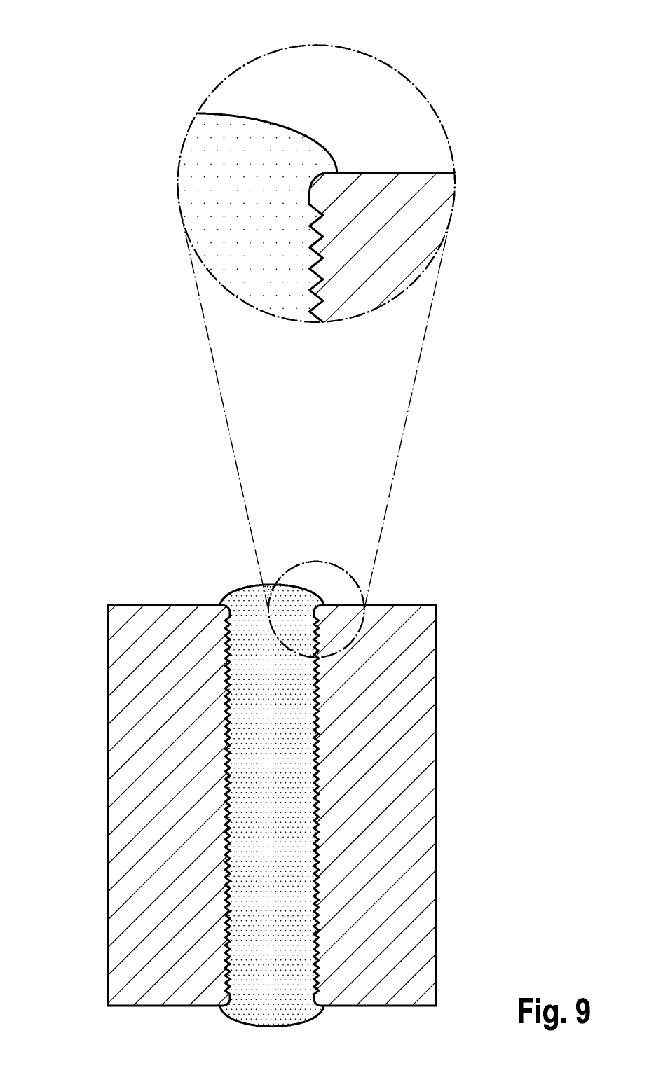

FIGS. 2 through 7 disclose further examples of embodiments of alternative forms and variants which may be assumed by a slotted passage in the stent according to the invention.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. Stent comprising a tubular base body having a lumen along a longitudinal axis, the base body having a plurality of circumferential support structures and one or more connectors, and two successive circumferential support structures being connected to one another via at least one connector, characterized in that one, multiple, or all support structures and/or connectors have a slotted passage, the slotted passage being filled with a radiopaque material and having a curvature which increases at least toward one end of the slotted passage.

2. Stent according to claim 1, wherein the slotted passage penetrates the outer surface of the support structure or of the connector which faces toward and faces away from the lumen of the stent, and extends parallel to the outer surface of the support structure or of the connector as an oblong opening having two opposite ends.

3. Stent according to claim 2, wherein the oblong opening in the slotted passage has a ratio of the maximum length l to the maximum width b of ≧2.

4. Stent according to claim 1, wherein the slotted passage has a curvature which increases toward both opposite ends of the slotted passage.

5. Stent according to claim 4, wherein the direction of the curvature of the two opposite ends is the same or different.

6. Stent according to claim 5, wherein the direction of the curvature over the progression of the slotted passage alternates multiple times, and preferably assumes a meandering shape.

7. Stent according to claim 4, wherein the shape of the curvature at the two opposite ends of the slotted passage is essentially uniform or oppositely directed.

8. Stent according to claim 1, wherein the shape of the curvature at one end or at the two opposite ends of the slotted passage has a curved, hooked, or spiral design.

9. Stent according to claim 1, wherein the filling with a radiopaque material forms a flat closure with the outer surface of the support structure and/or of the connector which surrounds the slotted passage.

10. Stent according to claim 1, wherein the filling with a radiopaque material forms a convex closure with the outer surface of the support structure and/or of the connector which surrounds the slotted passage, and the convex closure preferably has a design which projects beyond the rounded edge of the outer surface, and the edges of the outer surface of the support structure and/or of the connector particularly preferably are rounded.

11. Stent according to claim 1, wherein the radiopaque material contains or is composed of Au, Pt, and/or Ta, or a mixture or alloy thereof.

12. Stent according to claim 1, wherein the base body contains a metallic implant material or is composed of same.

13. Stent according to claim 1, wherein the base body contains a biocorrodible implant material or is composed of same.

14. Stent according to claim 1, wherein the base body contains a biocorrodible magnesium alloy or is composed of same.
